(19) United States
(12) Patent Application Publication
(10) Pub. No.: US 2022/0241115 A1
(43) Pub. Date: Aug. 4, 2022

(54) DERMAL PATCH

(71) Applicant: CARL FREUDENBERG KG, Weinheim (DE)

(72) Inventors: Birthe Lang, Weinheim (DE); Thomas Hofbauer, Hirschberg (DE); Bernd Schlesselmann, Weinheim (DE); Marc Pehr, Laudenbach (DE)

(21) Appl. No.: 17/616,210

(22) PCT Filed: May 19, 2020
(86) PCT No.: PCT/EP2020/063959

§ 371 Date: Dec. 3, 2021

(30) Foreign Application Priority Data
Jun. 4, 2019 (DE) 10 2019 115 005.1

(51) Int. Cl. A61F 13/02; D04H 1/425; D04H 1/4291; D04H 1/4334; D04H 1/435; D04H 1/4382; D04H 1/4391; D04H 1/544; D04H 1/549; D04H 1/55

(52) U.S. Cl. CPC A61F 13/0206; A61F 13/0289; D04H 1/425; D04H 1/4291; D04H 1/4334; A61F 2013/15024; D04H 1/43825; D04H 1/43918; D04H 1/544; D04H 1/549; D04H 1/55; D04H 1/435

(57) ABSTRACT

A dermal dressing is provided that comprises a foam layer and an adhesive layer applied directly thereon. A textile fabric is arranged on a side of the foam layer facing away from the adhesive layer, and the textile fabric comprises a vertically-lapped nonwoven. A method for producing the dermal dressing of the first aspect is also provided. The method comprises: providing the foam layer; applying the adhesive layer to the foam layer; applying the textile fabric to a side of the foam layer facing away from the adhesive layer; and bonding the foam layer and the textile fabric. The dermal dressing has optimal properties for its use in preventing pressure ulcers.

### CROSS-REFERENCE TO PRIOR APPLICATIONS

This application is a U.S. National Phase application under 35 U.S.C. § 371 of International Application No. PCT/EP2020/063959, filed on May 19, 2020, and claims benefit to German Patent Application No. DE 10 2019 115 005.1, filed on Jun. 4, 2019. The International Application was published in German on Dec. 10, 2020 as WO 2020/244924 under PCT Article 21(2).

### FIELD

Pressure ulcers (or decubitus wounds) have long been a significant problem in the areas of clinical as well as outpatient and inpatient care. For patients, such wounds are associated with strong pain, reduced quality of life, and increased morbidity. High treatment costs are incurred for health care—especially, due to prolonged hospital stays and intensive care requirements. In many countries, pressure ulcers occurring in hospitals and in the nursing facilities are already used as a quality characteristic, i.e., a high incidence rate indicates low quality in the care.

### BACKGROUND

Pressure ulcers arise from a combination of high or uneven pressure, shear forces, and friction in temporarily or permanently immobilized patients. The origin of these ulcers is usually located in the deeper tissue—especially, over angular bone projections such as the heel or sacral region. If the tissue is deformed by pressure and shear forces, an inflammatory response of the body starts, and ultimately leads to cell death and damage in the deeper tissue. This damage expands in a chain reaction to tissue layers at higher levels. They are often visible on the skin surface only after several days. The microclimate on the skin also contributes to the formation of pressure ulcers. Excessive moisture on the skin (e.g., sweat or urine) can result in increased friction and lead to degradation of the skin. Excessive dryness of the skin can in turn also lead to brittle skin, which is quickly injured.

Various standard measures are used for the prophylaxis of decubiti. These include the repositioning of patients, the use of pressure-relieving mattresses, and preventative skin care. Among other things, the prophylactic use of wound dressings and regular care and inspection of the skin can contribute significantly to the prevention of pressure ulcers.

EP1156838B1 describes a wound dressing that is primarily used for curative treatment, but also for prevention. The wound dressing consists of a hydrophilic foam layer, which is provided with an adhesive layer on the side facing the skin and with an absorbent layer on the opposite side. A disadvantage of using this dressing is that the dressing is primarily configured for curative treatment, i.e., for absorbing and retaining wound exudate, and thus the function is not optimized for prevention. In other words, there is no layer optimized for pressure distribution.

U.S. Pat. No. 7,182,085 A1 describes a wound dressing consisting of an absorbent part and a non-absorbent pressure-distributing part, which is targeted at the absorption of wound exudate and at the same time ensures a pressure distribution of static and dynamic pressure effects. A disadvantage of the wound dressing is that it has a very complicated structure, because it must meet the requirements for absorption as well for pressure distribution. Moreover, it cannot be cut.

U.S. Pat. No. 9,877,872 B2 also describes a wound dressing for curative treatment, which, in addition to an absorption layer, is provided with a designated pressure distribution layer in the form of a spacer fabric/knit fabric or woven fabric for decubitus prevention. The disadvantage here is the design in the form of a so-called island dressing, which, on the one hand, does not allow reuse of the dressing after an inspection of the skin, because the film edges easily adhere, and, on the other, is not optimized for all body regions. In addition, the film side impermeable to microorganisms, which is not required when used on intact skin, limits the moisture removal.

US 20180008476 A1 describes a wound dressing having properties optimized for decubitus prevention in the sacral region. The wound dressing also has tabs on the dressing which facilitate easy detachment from and re-attachment to the skin, and thus facilitate skin inspection. Furthermore, the dressing is characterized by different tensile strengths in the x- and y-directions. In the y-direction, a higher tensile strength is present in order to protect cells against deformation when the patient slides down in the bed. In the x-direction, the dressing is characterized by a higher stretchability in order to compensate for forces produced when the patient is being turned. It is disadvantageous here that the shape is suitable/optimized only for the sacral region and cannot also be efficiently used by, for example, cutting it to fit other body regions because the tensile expansion behavior is matched to the region. The shape and size cannot be individualized for individual patients.

An adhesive wound dressing for the sacral region that is to some extent adaptable in shape is described in, for example, EP1320343B1. Because of the “island dressing” shape, cutting is also not possible here.

WO 2017095460 A1 describes a hydrophilic polymeric matrix having closed cells for direct contact with the skin in order to distribute pressure and regulate moisture. The dressing consists of a layer and can therefore be tailored, i.e., individualized, to the patient. The wound dressing aims at ensuring healing, i.e., absorption and prevention, combined in one layer. This inevitably leads to compromising the pressure-distributing properties for the benefit of increased absorption capacity, which is, however, not necessary for purely prophylactic use.

Furthermore, applications are known which are aimed solely at prevention. EP 3162330 A1 describes an anatomically-shaped dressing for the protection of the sacral region, consisting of a moisture barrier layer and a bonding layer facing the skin. Although targeted moisture management on the skin can reduce the formation of pressure ulcers, it is insufficient by itself, because no directed pressure distribution takes place. In addition, the shape of the dressing is suitable only for the sacral region. Trimming or adaptation for other body regions is not possible efficiently.

US 20170087002 A1 describes a pressure-distribution dressing consisting of a pressure-distribution layer—preferably a cellular foam structure, a thin film, and a detachment layer for preventing pressure ulcers—especially in the face—when medical equipment is being used. A polyurethane foam with defined density, which is applied to a thin hydrophilic film with a release liner, is used as the pressure distribution layer. The dressing can be trimmed and adapted to the patient. The disadvantage here is that the pressure distribution takes place only by means of the foam. It can be assumed that the cells in the foam collapse under high pressure—especially in highly-loaded regions, such as the sacral region—and do not offer sufficient protection. This applies, especially, when the foam is moist. Furthermore, it is questionable whether a thin hydrophilic film has enough capacity to be able to quickly transport perspiration, for example, away from the skin.

WO 2017039668 A1 describes a product for preventing pressure ulcers comprising a substrate having an inner substrate surface with a substrate region and an opposite, outer substrate surface; wherein the opposite, outer substrate surface has a dynamic coefficient of friction in the range of 0.1 to 0.6; a bonding layer arranged on the inner substrate surface; a cushion layer having a surface facing the body, comprising a cushion layer region and an opposite surface facing the substrate; and an adhesive layer arranged on the surface facing the body; wherein the cushion layer is arranged between the bonding layer and the adhesive layer, and wherein the product, for preventing pressure ulcers, has a water-vapor permeability in the range of 2,400 g/m² per day to 10,000 g/m² per day. A foam or nonwoven material, e.g., an undulate nonwoven material, can be used as the cushioning layer. A disadvantage of the product is that the cushioning layer is separated from the skin only by the adhesive layer. In the case of a thin adhesive layer, skin irritations can be caused by fibers penetrating through the adhesive layer when a nonwoven material is used. In the case of a thick adhesive layer, the water-vapor permeability is again impaired.

WO 2018/007093 A1 discloses a dermal dressing comprising an open-cell foam layer and an adhesive layer applied thereon and intended for contact with the skin, wherein at least the side, facing the adhesive layer, of the foam layer has macropores whose cavities are spanned at least partially by a barrier layer formed from the foam layer. The dermal dressing is exceptionally well-suited for moisture regulation in existing (chronic) wounds—especially, for producing a moist wound climate, and consequently for moist wound treatment—especially of chronic wounds. However, the dermal dressing is not optimized for use in preventing pressure ulcers.

Commercially available dressings are, for example, Mepilex® Border (Mölnlycke® Healthcare) and Allevyn Life (Smith & Nephew). Both have a silicone-coated foam for adhesion to the skin and a nonwoven layer containing a superabsorbent. In addition, the Allevyn Life product has a pressure-distributing layer in the form of a spacer fabric. A disadvantage of both products is that they have a comparatively thick silicone layer (between 200 μm and 300 μm) and a significant amount of superabsorbent, which increases the costs of the products.

### SUMMARY

In accordance with a first aspect of the the present disclosure, a dermal dressing is provided that comprises a foam layer and an adhesive layer applied directly thereon. A textile fabric is arranged on a side of the foam layer facing away from the adhesive layer, and the textile fabric comprises a vertically-lapped nonwoven.

In accordance with a second aspect of the present disclosure, a method for producing the dermal dressing of the first aspect is provided. The method comprises: providing the foam layer; applying the adhesive layer to the foam layer; applying the textile fabric to a side of the foam layer facing away from the adhesive layer; and bonding the foam layer and the textile fabric.

The aim of the present disclosure is to provide a dermal dressing which has optimal properties for its use in preventing pressure ulcers. The dermal dressing shall thus be able to effectively reduce compressive stresses occurring in tissue and be suitable for regulating the moisture near the skin. In addition, the aforementioned disadvantages are to be at least partially eliminated.

This aim is achieved by a dermal dressing comprising a foam layer and an adhesive layer applied directly thereon and intended for contact with the skin, wherein, on the side, facing away from the adhesive layer, of the foam layer, a textile fabric is arranged, wherein the textile fabric is a vertically-lapped nonwoven.

### BRIEF DESCRIPTION OF THE DRAWINGS

Embodiments of the present disclosure will be described in even greater detail below based on the exemplary figures. The invention is not limited to the exemplary embodiments. Other features and advantages of various embodiments of the present disclosure will become apparent by reading the following detailed description with reference to the attached drawings which illustrate the following:

FIG. 1 illustrates the force-elongation behavior of the dermal dressing, in accordance with an embodiment; and

FIG. 2 shows the change in the contact surface for the body part to be protected with increasing weight force for a dermal dressing, in accordance with an embodiment.

### DETAILED DESCRIPTION

Surprisingly, it has been found that a dermal dressing according to the embodiments described herein is very well suited for preventing pressure ulcers, since it can efficiently reduce compressive stresses occurring in tissue. Without specifying a mechanism according to the disclosure, it is presumed that these advantageous properties of the dermal dressing are caused by the fact that the vertically-lapped nonwoven has fibers with a component that is vertical to the surface of the dermal dressing—in contrast to nonwovens that are produced using conventional nonwoven fabric formation methods (for example, carding with and without crosslapping, deposition from the air stream (Airlay and Airlaid), or wet-laid methods) and comprising fibers which are predominantly deposited horizontally. As a result, the dermal dressing can absorb and distribute pressure loads that occur especially well due to the vertically-oriented fibers, because the vertically-oriented fibers counteract compression of the dermal dressing, and the load surface of the body part to be protected is thus increased. Stresses occurring in the fabric can thereby be reduced. This applies especially to load peaks and has the consequence that the cells in the tissue are effectively protected from excessive loads, and thus the mechanisms leading to decubitus are attenuated.

A further advantage is that the foam layer constitutes a barrier layer for the skin and can thereby prevent fibers from coming into contact with the skin and leading to skin irritations. In addition, foam layers can be produced in a simple manner with a flat surface, which—compared to nonwoven layers, for example—allows the application of an adhesive layer, even in clearly-defined patterns.

A further advantage of the dermal dressing is that it has a high water-vapor permeability (MVTR)—presumably due to the capillary action of its vertically-oriented fibers. This is advantageous because excessive accumulation of moisture on the skin can thus be prevented.

It is also advantageous that the dermal dressing can be produced as rolled material and can therefore be cut, i.e., the shape can be ideally adapted to the patient.

In the case of a vertically-lapped nonwoven, the vertical component results from its specific production process. In contrast to a classical needling method, in which, as a rule, only a local vertical alignment of a horizontal fibrous web is achieved in the puncture channels of the needles, during the production of a vertically-lapped nonwoven, the entire formed fibrous web is generally folded in the vertical direction and is thus oriented vertically to the fibrous web plane. The angle of the folded fibrous web to the plane of the not-yet-folded web is preferably at least 20°, more preferably at least 60°, even more preferably about 90°, and/or at least 90°. The fibers or the fibrous web do not have to follow a single straight line or plane, but can, for example, be curved or follow a zig-zag course. Exemplary production methods suitable for vertically-lapped nonwovens can be found in the documents, US 2016/0244895 A1 (V-Lap method), U.S. Pat. No. 8,357,256 B2 (Wavemaker Santex method), and CN 104805597 (Anyou method).

The vertically-lapped nonwoven can contain a wide variety of matrix fibers. Matrix fibers are to be understood as fibers which are not present, or are present only to a small extent, as thermally-fused in the vertically-lapped nonwoven. The matrix fibers are preferably thermoplastic matrix fibers, which preferably have a melting point of the lowest-melting fiber component of greater than 100° C., e.g., of 100 to 200° C., and more preferably greater than 200° C. In one embodiment, the matrix fibers consist of one fiber component. In a further preferred embodiment, the matrix fibers consist of several fiber components. Full-profile fibers, multilobal full-profile fibers, hollow fibers, full-profile bicomponent fibers (e.g., core/sheath, side-by-side) are especially preferred. If the vertically-lapped nonwoven has binding fibers, it is advantageous if the melting point of the fiber component with the lowest melting point contained in the matrix fiber is above the melting point of the fiber component having the highest melting point contained in the binding fiber. The melting point of the fiber component having the lowest melting point in the matrix fiber is preferably at least 10° C.—especially preferably, at least 30° C.—above the melting point of the fiber component having the highest melting point in the binding fiber. Suitable polymer classes for producing the matrix fibers may be, among others, polyesters, polyamides, polyolefins, polyacrylonitrile, cellulose, or polyvinyl alcohols.

Especially suitable matrix fibers are hydrophobic fibers, i.e., fibers that are produced from polymers whose surface energy is less than 50 mJ/m². Especially suitable hydrophobic fibers are polyester and/or polyolefin fibers. It is advantageous for hydrophobic fibers that they do not absorb moisture, but can transport it away from the body along the z-oriented structure. This makes it possible to prevent the moisture from negatively influencing the structural properties of the nonwoven—especially, the compression properties.

The proportion of matrix fibers in the vertically-lapped nonwoven is preferably at least 20 wt %, e.g., from 20 to 100 wt %, more preferably at least 25 wt %, e.g., from 25 to 80 wt %, and more preferably at least 30 wt %, e.g., from 30 to 70 wt %, in each case relative to the total weight of the vertically-lapped nonwoven.

In a further preferred embodiment of the invention, the vertically-lapped nonwoven has binding fibers. Binding fibers are fibers that are at least partially fused in the vertically-lapped nonwoven and thereby create binder points between the fibers. Preferably, at least one fusible fiber component of the binding fiber—especially, an externally-arranged, fusible fiber component—has a melting point which is lower than the melting point of other fiber components contained in the nonwoven material, and, especially, lower than the melting point of the lowest-melting fiber component of the matrix fibers.

If the binding fiber has several fusible fiber components, the melting point of the highest-melting fiber component of the binding fiber is preferably more than 10° C.—especially preferably, more than 30° C.—below the melting point of the lowest-melting fiber component of the matrix fibers. Suitable binding fibers have a melting point of the highest-melting fiber component of below 250° C., e.g., from 100 to 200° C., more preferably below 180° C., e.g., from 100 to 180° C., and, especially, below 175° C., e.g., from 100 to 175° C.

Preferred fusible fiber components in binding fibers are polyolefin, polyester, polyamide, and/or mixtures thereof, as well as copolymers such as ethylene-vinyl acetate copolymers. Likewise preferred binding fibers are bicomponent fibers—especially, bicomponent fibers which contain polyolefin, polyester, ethylene-vinyl acetate copolymers, polybutylene terephthalate, and/or mixtures thereof as externally-arranged fiber components. The binding fibers can have different cross-sectional geometries, such as full-profile fiber, multilobal full-profile fiber, hollow fiber, and full-profile component fiber (e.g., core/sheath, side-by-side) geometries. Melt-binding fibers in the form of solid profile fibers are preferred.

In a preferred embodiment, the matrix fibers and/or the binding fibers are crimped fibers. The advantage of crimped fibers is that they impart improved recoverability to the textile fabric. This means that the textile fabric can maintain its volume even under strong mechanical load and thereby ensure increased pressure stability. Suitable crimped fibers are two-dimensional crimped fibers such as, for example, the “Grisuten” polyester fibers from Märkische Faser GmbH in Premnitz. Especially suitable are three-dimensional crimped fibers—especially, three-dimensional, crimped, binding fibers (e.g., spiral-crimped fibers)—because they have increased recoverability compared to the two-dimensional crimped fibers. The three-dimensional crimp can already be generated during the spinning process, e.g., like the “Softflex HY” of Indorama, or produced by thermal application of a bicomponent fiber, having, for example, side-by-side or eccentric, core-sheath, cross-sectional geometry, e.g., like the “EMF” fiber from Huvis.

The proportion of the crimped fibers—especially, of the three-dimensional, crimped, binding fibers—in the vertically-lapped nonwoven is preferably at least 20 wt %, e.g., from 20 to 100 wt %, more preferably at least 25 wt %, e.g., from 25 to 80 wt %, and more preferably at least 30 wt %, e.g., from 30 to 70 wt %, in each case relative to the total weight of the vertical nonwoven.

In a preferred embodiment, the crimped fibers have a number of crimp bends of 4 to 25 bends/cm—preferably between 6 and 18 bends/cm, and more preferably between 8 and 14 bends/cm.

In a further preferred embodiment, the vertically-lapped nonwoven is thermally-bonded. It is, further, preferably not needled. The needling is specifically disadvantageous in that it can interfere with the vertical orientation, present in the vertically-lapped nonwoven, of the fibers.

The vertically-lapped nonwoven may also contain, in addition to the crimped fibers, further, non-crimped fibers, e.g., non-crimped fibers containing polyesters, polyolefin, polyamide, and/or mixtures thereof. The further fibers are preferably present as staple fibers.

The fibers contained in the vertically-lapped nonwoven—especially, the matrix fibers, binding fibers, and/or the other fibers—are preferably staple fibers, preferably with a staple length between 20 and 150 mm, more preferably between 30 and 90 mm, and, especially, between 40 and 70 mm.

The fiber titer of the fibers contained in the vertically-lapped nonwoven—especially, of the matrix fibers, binding fibers, and/or of the further fibers—is preferably in the range of 0.9 to 100 dtex (g/10,000 m); more preferably, it is between 1.5 and 30 dtex, and, especially, between 3 and 11 dtex.

The vertically-lapped nonwoven preferably has an average thickness, measured in accordance with DIN EN ISO 9073-2:1997-02, of at least 1.5 mm, e.g., 1.5 mm to 15 mm, more preferably at least 2 mm, e.g., 2 mm to 10 mm, and/or 4 mm to 10 mm, and/or 2 mm to 5 mm, and/or 5 mm to 8 mm.

A wide variety of foams—especially, polymer foams—can, according to the invention, be used as the foam layer. The foam layer is preferably based upon polyurethane foam, e.g., polyether polyurethane or polyester polyurethane foam, polyether ester polyurethane foam, polyvinyl acetate foam, polyvinyl alcohol foam, or upon mixtures of these foams. The term, “based upon,” means more than 50 wt %, more preferably more than 70 wt %, and, especially, more than 90 wt %, relative to the total weight of the foam layer.

Especially preferably, the foam layer is based upon a hydrophilic polymer foam, i.e., a foam with an absorption time for water drops of less than 1 minute, preferably of less than 40 seconds, more preferably of less than 10 seconds, and more preferably of less than 1 second. Likewise preferable is a polymer foam which is produced from hydrophilic polymers—preferably, hydrophilic polyurethanes, and, especially, hydrophilic polyurethanes—as described in WO2018/007093. Hydrophilic polymer foams have the ability to absorb and store liquids. Compared to hydrophobic polymer foams, hydrophilic polymer foams thus have the advantage that they quickly absorb liquid from the skin surface and thus positively contribute to the microclimate on the skin. This is especially advantageous in the system, because the foam layer is separated from the skin only by the adhesive layer. Very particular preference is given to a polyurethane foam, because it combines a high degree of hydrophilicity with good elasticity and retention.

In a preferred embodiment, the foam layer is bonded to the textile fabric by means of a binder. Preferred binders are adhesive nonwovens, adhesive lattices, and/or hot-melt adhesives. The binders may contain co-polyamide, co-polyester, polyolefin, polyvinyl alcohol (PVA), ethylene vinyl acetate (EVA), thermoplastic polyurethane (TPU), polycaprolactone, terpolymers, and/or mixtures thereof. The binder preferably contains the aforementioned polymers in an amount of more than 50 wt %—especially preferably, of more than 70 wt %, and, in particular, more than 90 wt %—relative in each case to the total weight of the binder. With these binders, a good bond between the foam layer and the textile fabric can be achieved without negatively influencing the moisture transport. Especially preferred hot-melt adhesives are thermoplastic hot-melt adhesives—especially, hot-melt adhesives based upon polycaprolactone. This is advantageous in that good adhesion can be achieved without impairing the flexibility of the dermal dressing and thus the adaptability to body contours.

The hot-melt adhesive can be applied by a wide variety of methods known to the person skilled in the art—for example, by means of scattering, spraying, nozzle application, or slot application. Scattering is especially preferred because this creates punctiform bonds that allow a flexible and water-vapor-permeable bond.

In an especially preferred embodiment of the invention, the binder is applied point-by-point between the foam layer and the textile fabric. This is advantageous because good water-vapor permeability and flexibility can thereby be maintained.

The foam layer preferably has an average thickness, measured using a calibrated thickness gauge, of at least 0.3 mm, e.g., of 0.3 mm to 12 mm, more preferably of at least 0.4 mm, e.g., of 0.4 mm to 12 mm, even more preferably of 0.5 mm to 10 mm, more preferably still of 1 mm to 8 mm, and, especially, of 1 mm to 7 mm.

According to the invention, the dermal dressing has an adhesive layer. This may contain the most diverse materials known to the person skilled in the art, insofar as they are both dermatologically compatible and achieve a sufficient adhesive effect, to prevent the dermal dressing from slipping or detaching. Suitable adhesives are, for example, silicone, acrylic, and/or acrylate-based adhesives, wherein “based” is to be understood to mean more than 50 wt %, more preferably more than 70 wt %, and, especially, more than 90 wt %, relative to the total weight of the adhesive layer. Hydrogels, hydrocolloids, polyurethane gels, and/or rubber-based adhesives are likewise suitable. The adhesive layer is preferably a layer comprising a silicone layer—preferably a layer that contains more than 50 wt %, more preferably more than 70 wt %, and, especially, more than 90 wt % silicone, relative to the total amount of the adhesive layer. An adhesive layer comprising silicone as described, for example, in WO2018/007093 A1 is especially preferred. An advantage of silicone is that it is a very soft adhesive which can adapt well to the body contour. Its softness and elasticity additionally allow good absorption of shearing forces that may arise. Furthermore, silicone is a very skin-friendly adhesive which can be easily and gently detached from the skin without destroying skin cells or causing pain.

The adhesive layer may cover the entire surface of the foam layer. This is advantageous in that a high degree of adhesion to the skin is achieved, and thus slippage or detachment of the dermal dressing is prevented—for example, when positioning patients. Alternatively, the adhesive layer may cover only a part of the foam layer and be in the form of a pattern, e.g., waves, dots, strips, and/or as a grid-like pattern. This is advantageous in that the adhesion to the skin can be controlled selectively. This is advantageous, for example, for especially sensitive skin, as can be found, for example, in older patients (so-called parchment skin). The degree of coverage of the foam layer with the adhesive layer is preferably less than 90%, e.g., between 50% and 90%, and more preferably between 60% and 80%. The degree of coverage can be determined by means of visual assessment.

In a preferred embodiment, the adhesive layer has a thickness of less than 200 μm and/or is present in an application quantity of less than 200 g/m². This can be realized, for example, with the procedure described in WO 2018/007093. This is advantageous in that, due to the small thickness and in some cases partial coating of the adhesive layer, a higher water-vapor permeability and lower peel forces can be realized, wherein the latter enables a removal of the dressing that is gentle on the skin.

In a further preferred embodiment, the dermal dressing has an absorption time for water drops of less than 60 seconds, more preferably of less than 30 seconds, and even more preferably of less than 15 seconds. This is advantageous in that any liquid is quickly removed from the skin, and thus the accumulation of liquid is prevented.

In a further preferred embodiment of the invention, the dermal dressing has a thickness, measured in accordance with DIN EN ISO 9073-2:1997-02, of 3 to 20 mm, more preferably of 5 to 13 mm, and even more preferably of 8 to 10 mm. It has been found that, at these thicknesses, a good pressure distribution takes place, and the dermal dressing is simultaneously thin enough that it does not get in the way and also does not easily detach—for example, when the patient is being turned.

For some applications, it is advantageous if the dermal dressing is configured as rolled material. As a result, the user can freely select the desired shape and size as needed. Alternatively, the dermal dressing can already be present in the form prepared for the purpose of application—for example, as a protective dressing for the heel, back of the head, and/or sacral region.

It is conceivable for the dermal dressing to have a barrier layer—for example, a barrier film. The latter preferably consists of polyurethane or polyester, or mixtures thereof. The skin can be protected by the barrier layer against liquids and aggressive body exudates. The barrier layer is, expediently, water-vapor permeable; specifically, it preferably has a water-vapor permeability (MVTR value), measured in accordance with DIN EN 13726-2:2002-06, of at least 1,000 g/(24 h m²), preferably of at least 5,000 g/(24 h m²), and more preferably of at least 8,000 g/(24 h m²). This is advantageous in that an accumulation of moisture between the skin and the dermal dressing, which would lead to increased friction on the skin, can be prevented.

In a preferred embodiment, the barrier layer is arranged on the side, facing away from the foam layer, of the textile fabric. More preferably, the barrier layer has a thickness, measured in accordance with DIN ISO 23529:2010, of 10 to 70 μm—especially preferably, of 20 to 40 μm.

The dermal dressing is outstandingly well suited for hindering and/or preventing the development of pressure ulcers in temporarily or permanently immobilized patients. The disclosure is thus also directed to the use of the dermal dressing for this purpose. Preferred uses include the protection of bony body regions, such as the heel, back of the head, and sacral region, from pressure ulcers.

A further aspect of the present disclosure is to provide a method for producing the dermal dressing, comprising the following steps:

providing a foam layer;

applying an adhesive layer intended for contact with the skin to the foam layer;

applying a textile fabric to the side, facing away from the adhesive layer, of the foam layer, wherein the textile fabric is a vertically-lapped nonwoven; and

bonding of foam layer and textile fabric.

In a preferred embodiment, the foam layer and textile fabric are bonded by means of a binder. This bonding is preferably carried out by means of temperature and pressure.

The preferred embodiments discussed with respect to the dermal dressing also represent preferred embodiments with respect to the method.

Measuring methods: For the purposes of the present disclosure, the following measurement methods were used. In principle, in all measurement methods in which averages are formed, the person skilled in the art selects the number of values determined for averaging as a function of their scattering. The greater the deviations found, the more values they include in the determination.

Number of crimp bends—The number of crimp bends is understood to mean the number of half-sinusoidal arcs per cm. Each pronounced change in direction is referred to as a bend. Only bends that provide volume are counted. Individual fibers are placed in parallel on a collection board. The fibers must not be compressed or warped. A millimeter strip is applied to both sides of the fiber, which strip has a horizontal, linear marking at a distance of 20 mm. Place 1 brass plate on each of the marking lines (precisely-defined measurement length). Count the number of half-sinusoidal arcs on a crimped fiber length of 20 mm under the stereo microscope at 12-fold magnification, and record as the number of crimp bends in bends/cm. Each pronounced change in direction is referred to as a bend. Spiral-crimped fibers are read in the clamped state during crimping.

Absorption time for water drops—The time required for a water drop to be completely absorbed into the dermal dressing is measured as follows: The dermal dressing is laid flat with the silicone-coated wound contact layer. A water drop is applied using a pipette, and the time required for the water drop to enter the dermal dressing completely is timed. If the water drop has not been completely absorbed within 3 minutes, the absorption time must be assumed to be 180 seconds.

The thickness of the adhesive layer—The thickness of the adhesive layer is carried out by means of scanning electron microscopy with an acceleration voltage of 20 kV. In order to avoid charging effects and resulting measurement errors, the samples are sputtered with gold prior to the SEM examination. This takes place at an argon gas pressure of 0.1 mbar at a sputtering current of 30 mA at a distance of 10 cm. The sputtering time is 300 seconds. A fictitious reference surface is generated by placing a paper, coated on both sides with polyethylene, having a weight per unit area of 120 g/m2, on the adhesive layer. The thickness is determined as the distance between the underside of the paper and the lowest, adhesive-containing location at the respective measuring location. The evaluation takes place in cross-section by means of SEM. If it increases the contrast between an adhesive and foam layer, a backscatter detector is used. The thickness is measured in at least 10 locations evenly distributed over a range of at least 2 mm, and the average is determined. In order to avoid distortion by subsequent penetration of adhesive into the foam layer during formation of the cross-sectional area, the cut is made perpendicular to the side, facing away from the adhesive, of the foam layer.

Thickness of the foam layer—The foam thickness is measured at all four corners of a 10×10 cm sample using a calibrated thickness gauge. When measuring, care must be taken that the foam is not compressed. The measurement results are to be documented to two decimal places. The average foam thickness results from the average value of the four measurements.

The thickness of the textile fabric—The thickness of the textile fabric is measured in accordance with DIN EN ISO 9073 Part 2.

The embodiments of the present disclosure are explained in more detail below with reference to several examples.

### Example 1: Production of a Dermal Dressing According to the Invention with a Vertically-Lapped Nonwoven as Textile Fabric

A foam layer made of polyurethane (thickness 3 mm) according to the method described in WO 2018/007093 A1 is provided with a very thin silicone layer (20 g/m2 50% cover). Then, a vertically-lapped nonwoven having a density of 20 kg/m3 consisting of 60% polyester matrix fibers, 40% spiral-crimped binding fiber (“EMF” Huvis) is produced in 8 mm thickness according to the method described in CN104805597, applied to the foam layer, and thermally-bonded using hot-melt adhesive powder (140° C., 6 m/min) in a laminating system.

The dermal dressing obtained in this way has the properties shown in the following table:

| | | | | Adhesive | |
|---|---|---|---|---|---|
| | Total | Foam layer | Textile fabric | layer | |
| | thickness | thickness | thickness | thickness | Absorption |
| | [mm] | [mm] | [mm] | [μm] | time [s] |
| Dermal | 10 | 3 | 8 | 40 | 14 |
| dressing | | | | | |
| Mepilex | 4 | 1.6 | 0.2 + 2.2 | 300 | >180 |
| Border ® | | | | | |
| Allevyn | 7 | 1.7 | 3.7 + 1.6 | 250 | 24 |
| Life ® | | | | | |

### Example 2: Determination of Force-Elongation Behavior of the Dermal Dressing from Example 1

The force-elongation behavior of the dermal dressing from Example 1 is measured using a Zwick tensile tester as described below. A sample with a diameter of 25 mm is punched from the dermal dressing. The samples are inserted into the tensile tester and approached with a metal plate (diameter also 25 mm) with an initial load of 0.5 N, in order to ensure a defined starting point. The sample is then loaded with a force of 15 N (speed 5 mm/s) for 15 minutes. After relief, the sample is again briefly loaded with 25 N.

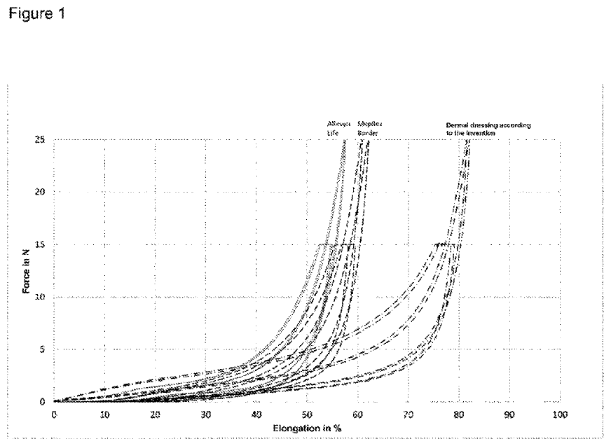

FIG. 1 illustrates the force-elongation behavior of the dermal dressing from Example 1 (dashed line) and compared to two, commercially available dermal dressings, Mepilex® Border (dotted line), Allevyn Life® (dot-dash line). It is found that the dermal dressing according to the invention has a higher elongation than the two, commercially available dermal dressings. The point of the curve at which the linear increase of the force with the elongation transitions into an exponential increase is crucial. From this point, the dressing is compressed to such an extent that buckling is possible only to a limited extent, and the stiffness increases sharply as a result of the compression that is present. It can be clearly seen that, for the dermal dressing according to the example, a higher expansion, and thus a higher penetration depth, is achieved at the same weight force. In the case of the commercially available dressings, Mepilex® Border and Allevyn Life®, the elongation at a weight force of 13 N is 54% and 51%, respectively, whereas the elongation is at about 76% for the dermal dressing according to the example. This is advantageous for the application for decubitus prevention, because this behavior increases the contact surface area for the body part to be protected, and thus the pressure is distributed over a larger surface area, thereby reducing load peaks.

### Example 3: Analysis of the Forces Occurring in Tissue when the Dermal Dressing According to the Invention is Used

Finite element modeling (FEM) was used to evaluate the dermal dressing according to the invention. Decubitus wounds, for the most part, arise in deeper tissue, rather than at the skin surface. Therefore, consideration purely of the pressure distribution at the surface is not sufficient for evaluating the effectiveness of dermal dressings. Finite element modeling was therefore used to evaluate the compressive stresses occurring in the fabric. Finite element modeling is based upon the use of numerical methods. A solid is divided into a finite number of parts, the physical behavior of which allows the behavior of the total body to in turn be calculated.

The method selected for evaluating the dermal dressing according to the invention was adapted according to A. Levy, M. B-O. Frank, and Amid Gefen, “The biomechanical efficacy of dressings in prevention wheels,” in Journal of Tissue Viability (2015) 24, 1-11. The biomechanical properties of the foot tissue were taken from Sara Behforootan, Panagiotis E. Chatzistergos, Nachiappan Chockalingam, Roozbeh Naemi, Journal of the mechanical behavior of biomedical materials 68 (2017) 287-295.

It is shown that, when the dermal dressing according to the invention is used at a weight force of 13 N, the maximum loads in the tissue (determined as a percentage as ξmax) at 18.6% are below the maximum loads in commercially available dressings (Mepilex® Border 23.3% and Allevyn Life® 21.9%). Compressive stresses occurring in this way can be reduced significantly more sharply compared to the commercially available dermal dressings.

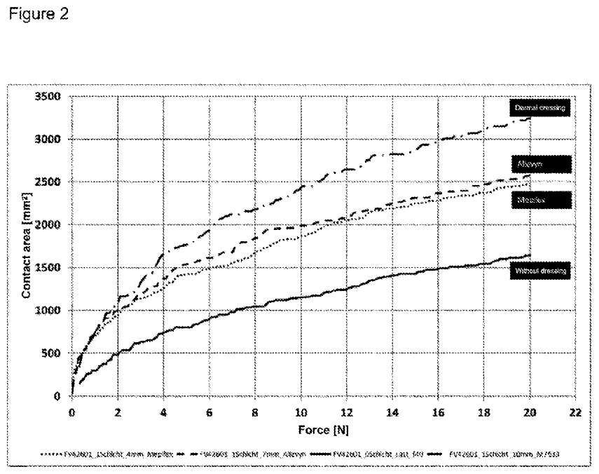

FIG. 2 shows the change in the contact surface for the body part to be protected (in the selected simulation, a foot) with increasing weight force for a dermal dressing (dashed line) compared to the commercially available products, Mepilex® Border (dotted line) and Allevyn Life® (dot-dash line). It is shown that the dermal dressing according to the example increases the contact surface by 100%, even under high forces, in comparison to the use of no dressing, whereas the commercially available products, Mepilex® Border and Allevyn Life®, increase the contact surface by only approximately 50%. Thus, when using the dermal dressing according to the example, the occurring pressure can be distributed over a larger area, and local load peaks can be reduced.

While the embodiments of the present disclosure have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present disclosure covers further embodiments with any combination of features from different embodiments described above and below.

Additionally, statements made herein characterizing the embodiments refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A dermal dressing comprising a foam layer and an adhesive layer applied directly thereon and suitable for contact with the skin, wherein a textile fabric is arranged on a side of the foam layer facing away from the adhesive layer, and wherein characterized in that the textile fabric comprises a vertically-lapped nonwoven.

2: The dermal dressing according to claim 1, wherein the vertically-lapped nonwoven comprises a matrix containing fibers.

3: The dermal dressing according to claim 2, wherein the proportion of fibers in the matrix of the vertically-lapped nonwoven is at least 20 wt %, relative to a total weight of the vertically-lapped nonwoven.

4: The dermal dressing according to claim 2, wherein the matrix of the vertically-lapped nonwoven contains binding fibers and/or contains bicomponent fibersas an external fiber component.

5: The dermal dressing according to claim 2, wherein the matrix of the vertically-lapped nonwoven contains crimped fibers.

6: The dermal dressing according to claim 1, wherein the vertically-lapped nonwoven is thermally-bonded.

7: The dermal dressing according to claim 1, wherein the vertically-lapped nonwoven has an average thickness, measured in accordance with DIN EN ISO 9073-2:1997-02, of at least 1.5 mm.

8: The dermal dressing according to claim 1, wherein the foam layer comprises a hydrophilic polymer foam.

9: The dermal dressing according to claim 1, wherein the foam layer is bonded to the textile fabric by means of a binder.

10: The dermal dressing according to claim 1, wherein the adhesive layer comprises silicone.

11: The dermal dressing according to claim 1, wherein the adhesive layer has a thickness of less than 200 μm and/or is present in an application quantity of less than 200 g/m2.

12: The dermal dressing according to claim 1, wherein the dermal dressing has a thickness, measured in accordance with DIN EN ISO 9073-2:1997-02, of 3 to 15 mm.

13: The dermal dressing according to claim 1, wherein the dermal dressings is designed as a rolled material.

14. (canceled)

15: A method for producing a dermal dressing according to claim 1, the method comprising:
providing the foam layer;
applying the adhesive layer to the foam layer;
applying the textile fabric to a side of the foam layer facing away from the adhesive layer; and
bonding the foam layer and the textile fabric.

16: The method according to claim 15, further comprising applying the dermal dressing to a patient to inhibit development of a pressure ulcer.

17: The dermal dressing according to claim 2, wherein the fibers are selected from polyester fibers, polyolefin fibers, polyamide fibers, cellulose fibers, and/or mixtures thereof.
